(19) United States
(12) Patent Application Publication
(10) Pub. No.: US 2009/0023630 A1
(43) Pub. Date: Jan. 22, 2009

(54) Methods and Means for Use in Diagnostics and Treatment of Diabetes

(76) Inventors: Jan Wouter Drijfhout, Leiden (NL); Bart Otto Roep, Leiden (NL)

Correspondence Address: MORGAN LEWIS & BOCKIUS LLP, 1111 PENNSYLVANIA AVENUE NW, WASHINGTON, DC, 20004, US

(21) Appl. No.: 11/883,417

(22) PCT Filed: Jan. 30, 2006
(86) PCT No.: PCT/NL2006/050017

§ 371 Date: Apr. 30, 2008

(30) Foreign Application Priority Data
Jan. 31, 2005 (EP) 05075234.4

(51) Int. Cl. A61K 38/10; C12Q 1/02; A61K 38/08; C12N 5/08; C07K 7/00; A61P 37/00

(52) U.S. Cl. 514/3; 530/303; 435/29; 435/375

(57) ABSTRACT

The present invention identified novel epitopes from the insulin B chain which is embodied in methods and means for diagnostics and treatment of type 1 diabetes. The epitopes were found in a peptide comprising a fragment of the human insulin B chain. Using HLA-A2 tetramers having the peptide of the invention cytotoxic T-cells were found in peripheral blood cells samples from healthy individuals. The invention demonstrates that these autoreactive CTL directed against insulin B chain are able to destroy insulin producing beta-cells. Moreover, a significant proportion of cytotoxic T-cells from islet transplant recipients with recurrent autoimmunity and loss of insulin production recognized this peptide or analogue thereof. The peptide has a high affinity for the HLA-A2 allele, in particular HLA-A*0201. Based on the novel epitopes according to the invention, diagnostic and therapeutic methods and medicaments for the prevention or treatment of type 1 diabetes are provided.

### FIELD OF THE INVENTION

The invention relates to the field of human disease, medicaments and diagnostics therefore. The invention in particular relates to diabetes.

### BACKGROUND OF THE INVENTION

Insulin-dependent diabetes mellitus (IDDM) is characterized by a T-cell mediated immune autoimmune destruction of the pancreatic beta cells, resulting in an irreversible loss of insulin production (1). The current inventors and others have demonstrated the presence of circulating islet autoreactive CD4+ T cells (1-5) around the clinical onset of disease, but evidence for a role of these T-cells in beta-cell destruction is lacking. The nonobese diabetic (NOD) mouse strain spontaneously develops diabetes in part owing to the activity of CD8+ T cells. This was first demonstrated by the finding that NOD stocks made deficient in MHC class I expression and CD8 T cells were completely Type 1 diabetes (T1D) resistant (6;7). Subsequent analyses indicated that MHC class I-dependent T cell responses are an essential component of both the initiation and progression of pancreatic beta cell destruction ultimately leading to T1D development in NOD mice (8;9).

Transgenic expression of HLA-A*0201 significantly accelerates T1D onset in NOD mice, with HLA-A*0201-restricted CD8 T cells appearing in early, prediabetic insulitic lesions. These results provide functional evidence that the HLA-A*0201 allele contributes to T1D development (10). The HLA-A*0201 allele has been shown to confer additional risk to the development of type 1 diabetes in patients who have the high-risk class II alleles HLA-DR3 and HLA-DR4 (11;12). Individuals who carried the HLA-A*0201 allele were more likely to develop autoantibodies and clinical type 1 diabetes before 5 years of age than individuals lacking HLA-A*0201 (12).

Autoreactive cytotoxic T-cells recognize peptide epitopes displayed on the beta cell surface in the context of HLA class I molecules. These 8-10 amino acid epitopes are considered derived primarily from beta cell proteins, but their identity remains largely unknown in humans (13; 14). Islet antigen-specific human CTL have only been documented twice, to a peptide from GAD (14) and to a peptide from islet amyloid polypeptide (IAPP or amylin) (13), but functional evidence for beta-cell cytotoxicity or association with the pathogenesis of type 1 diabetes is still lacking. In contrast, the majority of CD4+ infiltrating T cells recognize insulin and of these T cells, more than 90% react with the amino acids 9-23 of the insulin B chain in type 1 diabetes (15;16).

### SUMMARY OF THE INVENTION

The present invention identifies novel epitopes from the insulin B chain in type 1 diabetes. The epitopes were found in a peptide comprising amino acid residues 10-18 of the human insulin B chain. Using HLA-A2 tetramers having the peptide of the invention cytotoxic T-cells were found in peripheral blood cells samples from healthy individuals. We demonstrate that these autoreactive CTL directed against insulin B10-18 can destroy beta-cells. Moreover, a significant proportion of cytotoxic T-cells from islet transplant recipients with recurrent autoimmunity and loss of insulin production recognized this peptide or analogue thereof. The peptide has a surprisingly high affinity for the HLA-A2 allele, in particular HLA-A*0201. Based on the novel epitopes, the invention provides both diagnostic and therapeutic methods and medicaments for the prevention or treatment of type 1 diabetes.

### DETAILED DESCRIPTION OF THE INVENTION

In a first embodiment the invention therefore provides a peptide comprising amino acid residues 10-18 of the human insulin B chain. The peptide of the invention may comprise only amino acid residues 10-18 of the human insulin B chain, although such peptides may comprises further amino acids that are not from human insulin. The peptide may also consist of amino acid residues 10-18 of the human insulin B chain. Also provided is an analogue of the peptide of the invention having the same immunogenic properties in kind and not necessarily in amount, as a peptide of invention. Suitable analogues are peptide mimetics. Other analogues are peptide (-mimetics) having between 1 and 3 amino acid substitutions when compared to a peptide of the invention, preferably only 1 substitution and most preferably 1 conservative amino acid substitution. A conservative amino acid substitution is herein defined as a substitution scoring positive or zero according to the PAM 250 or Blosum 45 amino acid similarity matrices, which are known to the skilled person. An analogue of a peptide of the invention is capable of associating with HLA-A2 and is recognized by a T-cell specific for a peptide of the invention, when associated thereto.

The individual residues of the immunogenic insulin B chain protein derived (poly)peptides of the invention can be incorporated in the peptide by a peptide bond or peptide bond mimetic. A peptide bond mimetic of the invention includes peptide backbone modifications well known to those skilled in the art. Such modifications include modifications of the amide nitrogen, the α-carbon, amide carbonyl, complete replacement of the amide bond, extensions, deletions or backbone cross-links. See, generally, Spatola, Chemistry and Biochemistry of Amino Acids, Peptides and Proteins, Vol. VII (Weinstein ed., 1983). Several peptide backbone modifications are known, these include, ψ [CH2S], ψ [CH2NH], ψ [CSNH2], ψ [NHCO], ψ [COCH2] and ψ [(E) or (Z) CH═CH]. The nomenclature used above, follows that suggested by Spatola, above. In this context, ψ indicates the absence of an amide bond. The structure that replaces the amide group is specified within the brackets.

Amino acid mimetics may also be incorporated in the peptides. An “amino acid mimetic” as used here is a moiety other than a naturally occurring amino acid that conformationally and functionally serves as a substitute for an amino acid in a peptide of the present invention. Such a moiety serves as a substitute for an amino acid residue if it does not interfere with the ability of the peptide to elicit an immune response against the appropriate insulin B chain protein derived epitope. Amino acid mimetics may include non-protein amino acids, such as β-, γ-, δ-amino acids, β-, γ-, δ-imino acids (such as piperidine-4-carboxylic acid) as well as many derivatives of L-α-amino acids. A number of suitable amino acid mimetics are known to the skilled artisan, they include cyclohexylalanine, 3-cyclohexylpropionic acid, L-adamantyl alanine, adamantylacetic acid and the like. Peptide mimetics suitable for peptides of the present invention are discussed by Morgan and Gainor, (1989) Ann. Repts. Med. Chem. 24:243-252.

The invention further provides a proteinaceous complex comprising a peptide or analogue thereof of the invention. The complex preferably further comprises a protein either bound the peptide or covalently linked to said peptide or analogue thereof. When covalently linked the complex can comprise a fusion protein of the peptide or analogue thereof with an non-insulin B protein, or the complex may contain a chemical linkage of said peptide and said protein. In a preferred embodiment said complex comprises an HLA class I molecule or a peptide binding part, derivative and/or analogue thereof. Preferably, said HLA class I molecule is an HLA-A2 molecule or a functional part, derivative and/or analogue thereof. A functional part, derivative and/or analogue of an HLA-A2 molecule of the invention comprises the same peptide binding characteristics in kind, not necessarily in amount as said HLA-A2 molecule. Said part, derivative and/or analogue, preferably further comprises the same T-cell receptor binding capability as said HLA-A2 when associated with a peptide of the invention. A preferred HLA-A2 molecule is HLA-A*0201.

A functional part of an HLA-A2 molecule, which is also suitable for formation of tetramers, may lack the signal peptide, the transmembrane domain and the intracellular domain of HLA, e.g. amino acids 25-308, and this functional domain may be extended with an amino acid sequence which optionally can be biotinylated enzymatically. A functional derivative of an HLA-A2 molecule comprises at least 90% and preferably at least 95% and more preferably at least 99% homology to an HLA-A2 molecule. A functional analogue of an HLA-A2 molecule is a naturally occurring primate MHC-I molecule comprising similar peptide and T-cell binding characteristics in kind not necessarily in amount as an HLA-A2 molecule.

In a preferred embodiment said complex comprises a tetramer of a peptide or analogue thereof and an HLA-A2 molecule or a functional part, derivative and/or analogue thereof. As tetramers contain four copies, they may contain peptide of the invention, analogues of said peptide or mixtures thereof. Similarly, they may contain HLA-A2; a part, derivative and/or analogue thereof or both. Other multimers of peptide and HLA-A2, such as octa- and decamers are also provided by the invention. Preferably tetramers or other multimers may be labelled, for instance a fluorescent label, such as but not limited to FITC, TRITC, Rhodamine, GFPs etc., enzymatic moieties (Alkaline phosphatases, Peroxidase and the like), radioactive labels, metals (immunogold), immunolabels/tags or biotin and other labels known in the art and suitable for detection purposes.

With the discovery of specific CTLs that are involved in the destruction of beta-cells in the pancreas of individuals suffering from or at risk of suffering from type 1 diabetes, it is possible to target these specific T cells for treatment of the disease or reduction of the risk of developing the disease. One of such strategies is the targeting of toxins to these T-cells with the aim to at least reduce their number. This can be done in a non-specific way, however, considering side effects of such non-specific treatment this is preferably done by specifically targeting the T-cell that are specific for the peptide of the invention. Specific targeting utilizes the specificity of the T-cell receptor for the peptide when presented in the context of the HLA-A2 molecule. Thus the invention provides a method for at least reducing the number of CTL-cells, specific for a peptide of the invention presented in the context of an HLA-2 molecule, in a sample comprising providing said sample with a binding molecule capable of specifically recognizing said T-cell receptor. In a preferred embodiment said binding molecule comprises an HLA-A2 molecule or functional part, derivative and/or analogue thereof whereto a peptide or analogue thereof of the invention is bound. Preferably, said binding molecule is complex of the invention. The number of CTL can be reduced in a variety of ways. In a preferred embodiment said CTL number is reduced through providing said cells with a toxin that is directly or indirectly linked to said binding molecule. Preferably, said binding molecule and/or said complex further comprises a toxin such as, but not limited to, MMF, Alum or RicinA (preferably directly linked thereto).

The invention further provides a composition comprising a peptide or analogue thereof of the invention, or a complex of the invention. Said composition may be used as a pharmaceutical for the treatment of diabetes. Pharmaceutical compositions may be formulated according to procedures and using excipients known in the art, for instance in Remington: The Science and Practice of Pharmacy, 20^{th} Ed. (formerly called Remington's Pharmaceutical Sciences), Mack Publishing Co., 2000. Pharmaceutical compositions and formulations may be adapted by the skilled person or physician to facilitate the mode of administration (oral or injection), to suit the nature of the composition (peptide or proteinaceous complex) and the patients' condition.

The invention further provides a method for determining whether an individual has an immune response against insulin producing cells or is at risk of developing such an immune response. Said method comprises determining whether a sample comprising T-cells of said individual comprises T-cells that are reactive with a peptide of the invention. The individual can be an individual suffering from diabetes. The individual can also be a healthy individual, or a (future) recipient of a β-cell transplant. Islet transplantation provides a unique opportunity to monitor islet destruction in a relatively short window. The present invention demonstrates that elevated insulin B10-18 specific CTL precursor frequency in the peripheral blood of islet recipients precedes recurrent autoimmunity. This indicates that HLA-A2^{ins-B} tetramer staining is strongly correlated with detection of autoreactive CTLs and recurrent autoimmunity and graft failure after islet transplantation in type 1 diabetic patients. This indicates that elevated levels of insulin B10-18 specific CTLs in peripheral blood can be used as an indicator of early islet allograft loss, thereby facilitating efforts to preserve islet mass. The use of HLA-A2^{ins-B} (insertion B10-18) tetramers facilitates the prediction of islet allograft loss before the onset of clinical symptoms.

The method can also be used to monitor healthy individuals. The method is preferably used to monitor healthy individuals that are at risk of developing diabetes. The individual may be without (early) clinical symptoms of the disease but may be considered at risk because the individuals has one or more genetical or epidemiological risk factors such as, but not limited to, obesity. The method preferably comprises determining whether said sample comprises T-cells that recognize an HLA class I tetramer comprising a peptide of the invention or analogue thereof, the HLA tetramer preferably comprising HLA-A2 or a functional part, derivative and/or analogue thereof.

The invention further provides an isolated or recombinant human T-cell comprising a T-cell receptor specific for a peptide of the invention that is presented in the context of an HLA class I molecule. As it is currently common practise to clone specific T-cell receptors from T-cells, the present invention further provides a nucleic acid encoding a human T-cell receptor specific for a peptide of the invention. This nucleic acid can subsequently be used to generate cells that express the recombinant T-cell receptor. The invention thus further provides an isolated and/or recombinant cell provided with a T cell receptor specific for a peptide of the invention.

An autoreactive T-cell clone that is provided by the invention can be generated from PBMC of a healthy blood donor. Prior to the invention several attempts to isolate autoreactive CTL from recent onset type 1 diabetic patients have failed. Although this could have resulted from practical insufficiencies and low precursor frequencies, we have observed that peripheral CTL autoreactivity is suppressed at the time of clinical manifestation of disease, when the vast majority of beta-cells have been destroyed. In healthy donors, however, the immune system appears ignorant rather than tolerant (31), and this ignorance can be lost upon vigorous priming with dendritic cells as demonstrated here.

A peptide of the invention is capable of specifically associating with an HLA-A2 molecule. A non limiting, but preferred example of such an HLA-A2 molecule is HLA-A*0201. HLA-A*0201 is one of the most prevalent class I alleles, with a frequency of over 60% in type 1 diabetic patients (12). HLA-A*0201 molecules bind peptides that are 9 to 10 residues long. The anchor positions of an HLA-A*0201-restricted epitope are at positions 2 and 9, which have a strong affinity for hydrophobic amino acids. The best consensus anchor residues for position 2 are leucine, isoleucine, or valine. Relaxed criteria include methionine and even the neutral amino acid threonine as alternate possibilities for this position. At position 9, the best anchor residues are leucine and valine; whereas isoleucine, alanine, and methionine may be alternate possibilities (32-34). Thus an analogue of a peptide of the invention having between 1 and 3 amino acid substitutions is preferably substituted at an anchor position. The substitution is then of course with one of the mentioned other suitable anchor amino acids mentioned for HLA-A*0201 above.

The peptide of the invention comprises the insulin B chain amino acids 10-18: HLVEALYLV. This peptide perfectly fits HLA-A*0201. This was confirmed using an HLA binding assay. The HLVEALYLV peptide can be generated by immuno- and constitutive proteasomes. Proteasomes are the major proteases involved in generation of MHC class I-bound peptides. Proteasomes tend to generate the exact C-terminal ends of class I ligands, but are less precise at the N terminus, thus generating N-terminally extended precursors (35). We show that both immuno- and constitutive proteasomes generate the exact C-terminus of the insulin B10-18 peptide. Three natural HLA-A*0201 ligands, derived from the insulin B chain itself, were directly generated by the 20 S proteasomes in vitro, and distinct aminopeptidase(s) in the cytosol and ER will trim the N termini of these presented peptides to their appropriate size to allow binding to HLA-A2.

A number of methods can be used to induce antigen-specific tolerance for the prevention of type 1 diabetes. Several studies have shown that prevention of diabetes can be achieved in animal models by the down-regulation of autoreactive T cells after administration of immunodominant peptides derived from the major beta cell antigens. Prevention of diabetes in animal models has been achieved through oral, intranasal, intravenous or subcutaneous administration of B9 peptide from the insulin B chain (36); peptide B24-C36 from proinsulin (9;37); peptides from GAD65 (38-40); or peptides from the heat-shock protein hsp60 (41;42). The peptides can be administered several times if need be. Thus the invention provides a use of a peptide of the invention for the preparation of a medicament for at least delaying the onset of type 1 diabetes in an individual at risk of developing the disease or in a (future) recipient of a β-cell transplant. The invention further provides a method for at least delaying the onset of type I diabetes in an individual at risk of developing the disease, or a (future) recipient of a β-cell transplant comprising providing said individual with a peptide of the invention or an analogue thereof.

The invention further provides a method for the treatment of an individual having T-cells specific for a peptide of the invention comprising providing said individual with a proteinaceous complex of the invention, a binding molecule of the invention, or a composition of the invention. This at least in part reduces the number of CTLs specific for the peptide in the individual. The invention further provides a proteinaceous complex of the invention, a binding molecule of the invention, or a composition of the invention for the preparation of a medicament for the treatment of an individual suffering from, or at risk of suffering from, an immune response against insulin producing β-cells. In a preferred embodiment the disease is diabetes mellitus.

The invention further provides a proteinaceous complex of the invention, a binding molecule of the invention, or a composition of the invention, or a peptide of the invention for detecting CTL specific for a peptide of the invention. To this end the invention provides a diagnostic kit comprising a peptide of the invention or analogue thereof, a complex of the invention, a binding molecule of the invention or a composition of the invention. In a preferred embodiment said kit comprises an HLA-class I molecule or a peptide binding part, derivative and/or analogue thereof, preferably an HLA-A2 molecule peptide binding part, derivative and/or analogue thereof.

With the advent of stem cell technologies and the increasing possibilities for manipulating the differentiation thereof it is likely that it will become possible to provide diabetes patients with precursors of β-cells that will differentiate into mature β-cells in the transplanted individual. Thus when herein mention is made of a (future) recipient of β-cells, this includes (future) recipients of β-cell precursor cells that mature to β-cells in the grafted individual.

The present invention demonstrates that ignorance or tolerance to islet autoantigen can be lost upon in vitro priming with dendritic cells loaded with the autoantigenic peptide. In an embodiment of this invention this process can also be reversed such that activated CTL specific for a peptide of the invention are tolerized. To this end the invention provides a method for incubating dendritic cells for a peptide (and/or analogue thereof) of the invention comprising activating said dendritic cells in the presence of a glucocorticoid hormone and loading said dendritic cells with a peptide of the invention or an analogue thereof. Activation of dendritic cells may be accomplished by administration of CD40 ligand, CD40 binding antibodies or fragments/derivatives thereof, or by administration of LPS or polyI/C. The glucocorticoid may be dexamethasone, but also any other glucocortoid molecule capable of activating glucocorticoid receptors, such as beclomethasone, 6-methylprednisolone, dexamethasone, hydrocortisone and triamcinolone.

Such dendritic cells, when brought into contact with a CTL specific for a peptide of the invention, can tolerize this T-cell. Reference is made to WO 01/24818, incorporated herein by reference, and references 44 and 45, for detailed descriptions of methods for generating such dendritic cells and how they may be used to induce tolerance. It is for instance mentioned therein that the glucocorticoid preferably comprises dexamethasone or functional equivalent thereof. It is further described therein that the dendritic cells are preferably obtained through differentiation of peripheral blood monocytes. These and other preferences are therefore also include in the present invention.

The present invention further provides a method for obtaining dendritic cells that can tolerize a CTL specific for a peptide of the invention comprising contacting said dendritic cells with vitamin D3. Reference is made to reference 43 for a detailed description of methods for generating such dendritic cells using vitamin D3 and for methods for using such dendritic cells to tolerize CTL. Thus the present invention further provides a dendritic cell that loaded with a peptide or analogue thereof of the invention. In a preferred embodiment, said dendritic cell has been treated with vitamin D3 or a functional equivalent thereof, a glucocorticoid or a functional equivalent thereof or both. Further provided is a method for at least in part reducing the destruction of β-cells in an individual comprising providing said individual with a dendritic cell of the invention.

### BRIEF DESCRIPTION OF THE DRAWINGS

FIG. 1

In vitro proteasome-mediated digestions of the 30-mer insulin B chain peptide containing potential HLA-A*0201-restricted epitopes. 20S proteasomes isolated from an EBV-transformed B cell line and an HELA cell line were coincubated with 30-mer insulin B chain peptides at 37° C. for 20 hrs. Digestion mixtures were analyzed by mass spectrometry as described in Materials and Methods. The intensity is expressed as percentage of total summed mass-peak intensities of digested 30-mer. Predicted epitope used for CTL induction is printed in bold.

FIG. 2

Cytotoxic cytokine release by CTLs after stimulation with insulin B10-18. 50.000 CTLs, either untreated (open bars) or treated (black bars) with insulin B10-18, were cultured on anti-INFg, anti-granzyme B, or IL-10 antibodies coated culture plates, whereafter INFg, anti-granzyme B, and IL-10 release was measured by ELIspot analysis. The bars show the amount of cells producing IFNg, granzyme B, or IL-10. Data are expressed as means±SD.

*P<0.0005 compared to the untreated CTLs.

FIG. 3

Insulin B chain 10-18 specific CTL frequency in peripheral blood samples of islet allograft recipients who have persistent islet graft function versus recurrent autoimmunity.

### EXAMPLES

### Methods

### Epitope Prediction

Peptides of the human insulin β chain that potentially bind to HLA-A*0201 were predicted using the peptide binding motif and the software reported before (17; 18).

### Peptide Synthesis

Peptides were synthesized using Fmoc amino acids and PyBop/NMM chemistry. Synthetic peptides were analyzed by reversed phase HPLC (purity was at least 85%) and Maldi-T of mass spectrometry (expected masses were confirmed) (19;20).

### Peptide-HLA Binding

Peptide binding was assessed as described (21). In short, a mixture of 100 fmol Fl-labeled HLA-A*0201 binding peptide, 200 fmol recombinant HLA-A*0201 heavy chain, 15 μmol P2M and serial dilutions of the test peptides in a buffer of 100 mM sodium phosphate, 75 mM NaCl and 1 mM CHAPS (100 μl end volume) was incubated 24 h at room temperature. HLA-bound and non-bound Fl-labeled peptides were separated by HPLC gel permeation chromatography with fluorescence detection. The concentration at which the test peptide reduced the amount of HLA-bound Fl-labeled peptide by 50% (IC₅₀) was calculated using non-linear regression analysis.

### TAP Translocation Assays

The TAP-dependent translocation assay was performed as described elsewhere (22). In short, peptides of interest were tested for their ability to compete for TAP-dependent translocation of a 125′-iodinated model peptide in streptolysin O-permeabilized EL-4 cells.

Generation of HLA-A2-peptide tetramers Tetrameric HLA-A2-peptide complexes were prepared essentially as previously described (23). Briefly, recombinant HLA A2 and human 132 microglobulin, produced in Escherichia coli, were solubilized in urea and injected together with each synthetic peptide into a refolding buffer consisting of 100 mM Tris (pH 8.0), 400 mM arginine, 2 mM EDTA, 5 mM reduced glutathione, and 0.5 mM oxidized glutathione. Refolded complexes were purified by anion exchange chromatography using DE52 resin (Whatman, Tewksbury, Mass.) followed by gel filtration through a Superdex 75 column (Amersham Pharmacia Biotech, Piscataway, N.J.). The refolded HLA-B8-peptide complexes were biotinylated by incubation for 16 h at 30° C. with the BirA enzyme (Avidity, Denver, Colo.). Tetrameric HLA-peptide complexes were produced by the stepwise addition of extravidin-conjugated PE (Sigma, St. Louis, Mo.) to achieve a 1:4 molar ratio (extravidin-PE:biotinylated class I).

### In Vitro Proteasome-Mediated Digestions

20S Proteasomes were purified from a EBV-(immuno proteasome) and a HELA cell line (constitutive proteasome) as described (24). High LMP2 and LMP7 contents were confirmed by two-dimensional immunoblotting (data not shown). To assess kinetics, digestions were performed with different incubation periods. The insulin B chain peptides (30 mer, 20 μg) were incubated with 1 μg of purified proteasome at 37° C. for 2, and 20 h in 300 μl proteasome digestion buffer (25). TFA (30 μl) was added to stop the digestion and samples were stored at −20° C. before mass spectrometric analysis.

### Generation and Isolation of Insulin B Chain 10-18-Specific CD8+ T Cells

Human HLA-A2 homozygous peripheral blood mononuclear cells (PBMCs) were isolated by Ficoll gradient from HLA-typed buffy coats, obtained from healthy blood donors. Monocytes were cultured in RPMI 1640 medium (Gibco Life Technologies, Breda, The Netherlands) with 10% heat-inactivated FCS and p/s, supplemented with 1,000 units/ml IL-4 (Strathmann Biotec AG, Hannover, Germany), and 800 units/ml GM-CSF (Leucomax, Novartis Pharma, Arnhem, the Netherlands) for 6 days. At d 6, the cells were cultured on CD40L cells for 48 hr to induce DC maturation. Mature DCs were pulsed with 10 mg/ml insulin B chain 10-18 peptide for 2 h at 37° C. in serum-free AIM-V medium (GIBCO-BRL, Breda, The Netherlands). After washing, APC and responder cells (CD4-depleted autologous PBMC) (1:10) were cultured in 24-well culture plates. Culture medium was IMDM-supplemented with p/s, 10% human serum (HS), and 20 U/mL IL-2. The cells were kept at 37° C. in an humidified, 5% CO2 air mixture. At d 5, 20 U/mL of IL-2 was added. From day 7 on, the T-cell cultures were restimulated weekly with peptide-pulsed autologous DCs. The T-cell lines were expanded with 20 U/ml IL-2-containing culture medium. At d 21, insulin B chain 10-18-tetramer+CD8+ T cells were sorted by FACSO and seeded at 1 cell/well in 96-wells plates, each well containing 1×105 irradiated allogenic PBMCs, 5×10³ irradiated EBV-B cells and 5×10³ irradiated insulin B chain 10-18-pulsed EBV-B cells in IMDM with p/s, 10% HS and 20 U/ml IL-2. Growing CD8+ T cell clones were isolated and restimulated weekly as described above.

Cell Staining with MHC-Peptide Tetrameric Complexes

1×10⁶ T-cells were stained with 1 mg/mL PE-labeled HLA-A2ins-B tetramers (in 500 mL phosphate-buffered saline (PBS) with 10% FCS) and incubated for 20 min in dark at RT. Cells were washed with 200 mL PBS/1% FCS. After tetramer staining, cells were incubated with anti-CD8/APC and anti-CD3/FITC antibodies (Becton Dickenson, Alphen aan den Rijn, The Netherlands) in 200 mL PBS/1% FCS for 30 min at 4° C. After washing twice, cells were resuspended in PBS/1% FCS and analyzed in a FACSCalibur (Becton Dickenson, Alphen aan den Rijn, The Netherlands)

### IFN-γ and Granzyme B Production

To determine specific excretion of the cytokines IFN-γ, granzyme B, and IL-10, CTLs were harvested by gently rinsing the wells and washing the collected CTLs in a large volume of IMDM. CTLs were treated with or without the peptide and were subsequently plated on anti-cytokine (IFN-γ, granzyme B, and IL-10) antibody-precoated ELISA plates and cultured overnight in IMDM supplemented with 1% human serum at 37° C. 5% CO2. After lysis of the cells with ice-cold deionized water, the plates were washed with PBS/0.05% Tween-20 and incubated with biotinylated detector antibody for 1 h at 37° C., followed by a second incubation with gold-labeled anti-biotin antibody (GABA) for 1 h at 37° C. All antibodies were diluted in PBS/1% BSA. After extensive washing with PBS/0.05% Tween-20, the plates were developed according to the manufacturer's protocol (U-CyTech, Utrecht, the Netherlands). Spots were counted on an Olympus microscope and analyzed with Olympus Micro Image 4.0 software (Paes Nederland, Zoeterwoude, the Netherlands). Results are expressed as means±SD of triplicate wells.

HLA-A2ins-B Tetramer Staining in Patients Receiving Islets of Langerhans

We tested the presence of insulin B10-18-specific CTLs with the HLA-A2ins-B tetramer staining in PBMCs of 9 HLA-A*0201 positive patients that received human pancreatic islets (26;27). All were C-peptide-negative type 1 diabetes (>25 years) patients with a functioning renal allograft transplanted 2-6 years earlier. Patients were under different immunosuppressive regimens: 1) cyclosporin, azathioprin, and prednisolone (n=3); 2) cyclosporin, azathioprin, and prednisolone in combination with anti-thymocyte globulin (ATG) (n=3) or 3) cyclosporin, MMF, and prednisolone (n=3). PBMCs (collected from blood samples 1 to 50 weeks after transplantation) were isolated by Ficoll density centrifugation and screened for the presence of insulin B chain 10-18-specific CTLs. The presence of these CTLs was correlated with recurrent autoreactivity against other islet auto-antigens and clinical outcome.

### Results

### Example 1

### HLA Binding

### Identification of HLA-A*0201 Binding Peptides from the Insulin B Chain

To select candidate HLA-A*0201 binding peptides from the insulin B chain, the molecule was screened for HLA-A*0201 binding motif containing peptides with a combination of two known binding prediction algorithms (18). Only peptides of 9 to 11 aa length were included. In total, 15 peptides (5 nonamers, 5 decamers, and 5 11-mers) were synthesized in order to determine their actual binding affinity for HLA-A*0201 using a competition-based cellular binding assay (28). Ten high affinity binding peptides were identified (IC₅₀ 6 μM), and 2 peptides bound with intermediate affinity (6 μM<IC₅₀ 15 μM), whereas the other peptides displayed a low (15 μM<IC₅₀ 100 μM) or undetectable binding capacity (IC₅₀>100 μM), (Table 1).

TABLE 1

Binding Affinity for HLA-A*0201 of
9-mers, 10-mers and 11-mers derived
from the insulin B chain

| | | |
|---|---|---|
| Peptide* | Sequence | IC50** |

| | | |
|---|---|---|
| 5-15 | HLCGSGLVEAL | 0.44 |
| 10-20 | HLVEALYLVCG | 0.19 |
| 14-24 | ALYLVCGERGF | 2.26 |
| 8-18 | GSHLVEALYLV | 3.73 |
| 16-26 | YLVCGERGFFY | 8.59 |
| 10-19 | HLVEALYLVC | 1.75 |
| 9-18 | SHLVEALYLV | 1.37 |
| 14-23 | ALYLVCGERG | >100 |
| 5-14 | HLCGSHLVEA | 0.06 |
| 16-25 | YLVCGERGFF | 2.30 |
| 10-18 | HLVEALYLV | >0.01 |
| 11-19 | LVEALYLVC | 3.60 |
| 14-22 | ALYLVCGER | 90.24 |
| 4-12 | QHLCGSHLV | 9.12 |
| 9-17 | SHLVEALYL | 59.45 |

*) Position in the insulin B chain starting at the NH₂-terminal aa of the peptide.
**) IC₅₀ is peptide concentration needed to inhibit binding of FL-labeled reference peptide for 50% (IC₅₀ in μM).

### Example 2

### Proteasomal Cleavage

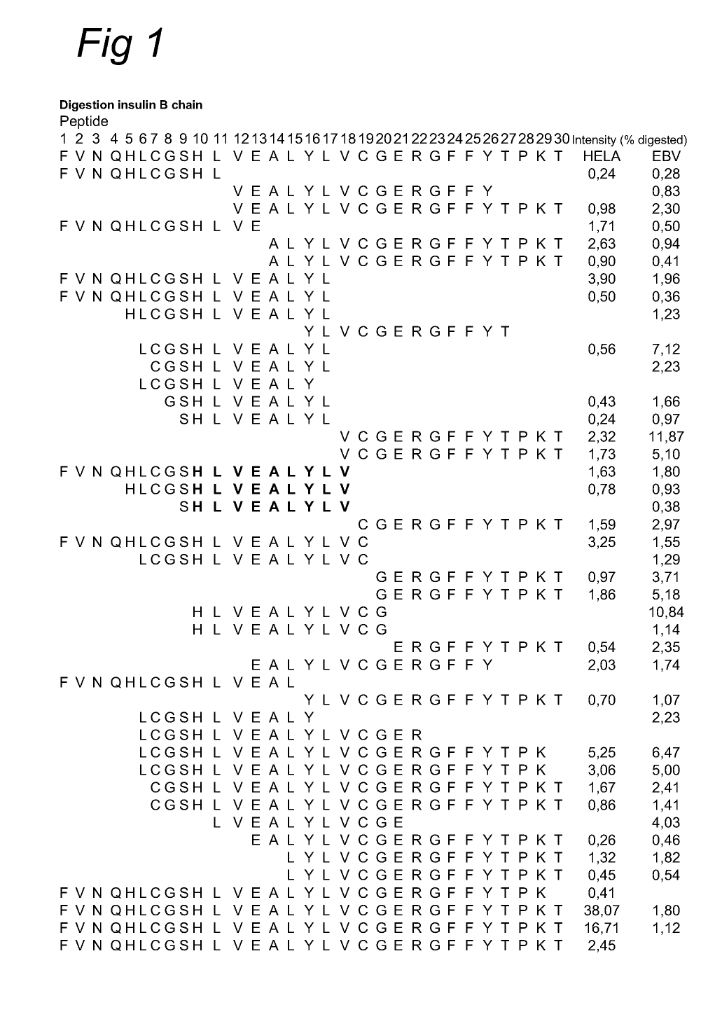

Dominant cleavage sites at the C-terminus were identified by in vitro digestion with immuno- and constitutive proteasomes. The insulin B chain was digested by immuno- and constitutive proteasomes that were isolated from an EBV-transformed B-cell line and the human cervical cancer cell line HELA, respectively. Digestion patterns of the insulin B chain, all containing potential high affinity HLA-A*0201 binding epitopes, are shown in FIG. 1. Both types of proteasomes showed three peptides (18, 14, and 10 amino acids long) that were processed with the exact C-terminus at position 18 of the insulin B chain, and two peptides (19 and 14 amino acids long) generated the exact C-terminus at position 20 of the insulin B chain (FIG. 1). The peptide identity was confirmed by tandom mass spectometry and sequencing. This indicates that both immuno- and constitutive proteasomes can generate peptides with strong binding affinity to HLA-A2

### Example 3

### Detection of Insulin B10-18 Specific CTL

The insulin B10-18 peptide was shown to be the best binder to HLA-A*0201 in an HLA-A2 assembly assay and this peptide is processed by both immuno- and constitutive proteasomes. PBMCs from a healthy donor were primed with this peptide in vitro and were tested for the presence of insulin B chain 10-18 specific CTLs. To facilitate the detection and quantification of insulin B10-18-specific HLA-A*0201 restricted CTLs, we constructed an HLA-A2ins-B tetramer. Blood cell samples were stained with the HLA-A2ins-B tetramer and then analyzed by flow cytometry. After several rounds of in vitro stimulation with insulin B10-18, more than 2% of the CD8 positive T-cells were positive for the HLA-A2ins-B tetramer.

### Example 4

### IFN-γ and Granzyme B Production

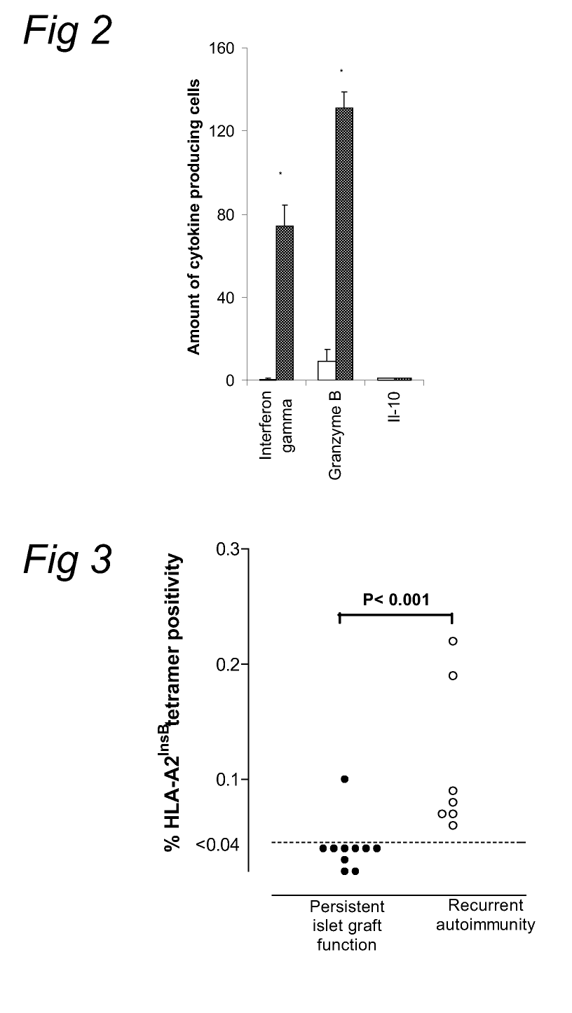

Activated CTLs are known to produce high levels of the cytokine IFNg and the cytotoxic enzyme granzyme B. We determined the number of IFNγ, granzyme B, and IL-10 producing CTLs by ELIspot analysis. CTLs produced high levels of IFNγ and granzyme B upon stimulation by the insulin B10-18 peptide (P<0.001). IL-10 was not produced by peptide specific CTLs. These results indicate that the CTLs show cytolytic activity upon stimulation by the insulin B10-18 peptide (FIG. 2).

### Example 5

### Detection of Insulin B Chain 10-18-Specific CTLs in Type 1 Diabetic Patients

We next addressed the issue of a possible in vivo correlation between presence of insulin B10-18-specific CD8+ T cells and recurrent autoimmunity after islet transplantation. We screened nine HLA-A*0201 positive islet-recipients for levels of insulin B10-18-specific CTLs in peripheral blood using ex vivo tetramer staining. HLA-A2ins-B tetramer staining revealed a strong correlation between detection of autoreactive CTLs and development of recurrent autoimmunity after islet transplantation and subsequent graft failure in type 1 diabetic patients (p<0.001) (FIG. 3).

### REFERENCES

1. Roep, B O: The role of T-cells in the pathogenesis of Type 1 diabetes: From cause to cure. Diabetologia 46:305-321, 2003
2. Roep, B O, Arden, S D, De Vries, R R, Hutton, J C: T-cell clones from a type-1 diabetes patient respond to insulin secretory granule proteins. Nature 345:632-634, 1990
3. Roep, B O, Kallan, A A, Hazenbos, W L, Bruining, G J, Bailyes, E M, Arden, S D, Hutton, J C, De Vries, R R: T-cell reactivity to 38 kD insulin-secretory-granule protein in patients with recent-onset type 1 diabetes. Lancet 337:1439-1441, 1991
4. Arif, S: Autoreactive T cell responses show proinflammatory polarization in diabetes but a regulatory phenotype in health. 2004
5. Arif, S, Tree, T I, Astill, T P, Tremble, J M, Bishop, A J, Dayan, C M, Roep, B O, Peakman, M: Autoreactive T cell responses show proinflammatory polarization in diabetes but a regulatory phenotype in health. J. Clin. Invest 113:451-463, 2004
6. Serreze, D V, Leiter, E H, Christianson, G J, Greiner, D, Roopenian, D C: Major histocompatibility complex class I-deficient NOD-B2mnull mice are diabetes and insulitis resistant. Diabetes 43:505-509, 1994
7. Wicker, L S, Leiter, E H, Todd, J A, Renjilian, R J, Peterson, E, Fischer, P A, Podolin, P L, Zijlstra, M, Jaenisch, R, Peterson, L B: Beta 2-microglobulin-deficient NOD mice do not develop insulitis or diabetes. Diabetes 43:500-504, 1994
8. Wong, F S, Visintin, I, Wen, L, Flavell, R A, Janeway, C A, Jr.: CD8 T cell clones from young nonobese diabetic (NOD) islets can transfer rapid onset of diabetes in NOD mice in the absence of CD4 cells. J. Exp. Med. 183:67-76, 1996
9. DiLorenzo, T P, Graser, R T, Ono, T, Christianson, G J, Chapman, H D, Roopenian, D C, Nathenson, S G, Serreze, D V: Major histocompatibility complex class I-restricted T cells are required for all but the end stages of diabetes development in nonobese diabetic mice and use a prevalent T cell receptor alpha chain gene rearrangement. Proc. Natl. Acad. Sci. U.S.A 95:12538-12543, 1998
10. Marron, M P, Graser, R T, Chapman, H D, Serreze, D V: Functional evidence for the mediation of diabetogenic T cell responses by HLA-A2.1 MHC class I molecules through transgenic expression in NOD mice. Proc. Natl. Acad. Sci. U.S.A 99:13753-13758, 2002

11. Fennessy, M, Metcalfe, K, Hitman, G A, Niven, M, Biro, P A, Tuomilehto, J, Tuomilehto-Wolf, E: A gene in the HLA class I region contributes to susceptibility to IDDM in the Finnish population. Childhood Diabetes in Finland (DiMe) Study Group. Diabetologia 37:937-944, 1994
12. Robles, D T, Eisenbarth, G S, Wang, T, Erlich, H A, Bugawan, T L, Babu, S R, Barriga, K, Norris, J M, Hoffman, M, Klingensmith, G, Yu, L, Rewers, M: Millennium award recipient contribution. Identification of children with early onset and high incidence of anti-islet autoantibodies. Clin. Immunol. 102:217-224, 2002
13. Panagiotopoulos, C, Qin, H, Tan, R, Verchere, C B: Identification of a beta-cell-specific HLA class I restricted epitope in type 1 diabetes. Diabetes 52:2647-2651,
14. Panina-Bordignon, P, Lang, R, van Endert, P M, Benazzi, E, Felix, A M, Pastore, R M, Spinas, G A, Sinigaglia, F: Cytotoxic T cells specific for glutamic acid decarboxylase in autoimmune diabetes. J. Exp. Med. 181:1923-1927, 1995
15. Daniel, D, Gill, R G, Schloot, N, Wegmann, D: Epitope specificity, cytokine production profile and diabetogenic activity of insulin-specific T cell clones isolated from NOD mice. Eur. J. Immunol. 25:1056-1062, 1995
16. Wegmann, D R, Norbury-Glaser, M, Daniel, D: Insulin-specific T cells are a predominant component of islet infiltrates in pre-diabetic NOD mice. Eur. J. Immunol. 24:1853-1857, 1994
17. Drijfhout, J W, Brandt, R M, D'Amaro, J, Kast, W M, Melief, C J: Detailed motifs for peptide binding to HLA-A*0201 derived from large random sets of peptides using a cellular binding assay. Hum. Immunol. 43:1-12, 1995
18. D'Amaro, J, Houbiers, J G, Drijfhout, J W, Brandt, R M, Schipper, R, Bavinck, J N, Melief, C J, Kast, W M: A computer program for predicting possible cytotoxic T lymphocyte epitopes based on HLA class I peptide-binding motifs. Hum. Immunol. 43:13-18, 1995
19. Teixeira, A, Benckhuijsen, W E, de Koning, P E, Valentijn, A R, Drijfhout, J W: The use of DODT as a non-malodorous scavenger in Fmoc-based peptide synthesis. Protein Pept. Lett. 9:379-385, 2002
20. Kessler, J H, Beekman, N J, Bres-Vloemans, S A, Verdijk, P, van Veelen, P A, Kloosterman-Joosten, A M, Vissers, D C, ten Bosch, G J, Kester, M G, Sijts, A, Wouter, D J, Ossendorp, F, Offringa, R, Melief, C J: Efficient identification of novel HLA-A(*)0201-presented cytotoxic T lymphocyte epitopes in the widely expressed tumor antigen PRAME by proteasome-mediated digestion analysis. J. Exp. Med. 193:73-88, 2001
21. Ottenhoff, T H, Geluk, A, Toebes, M, Benckhuijsen, W E, van Meijgaarden, K E, Drijfhout, J W: A sensitive fluorometric assay for quantitatively measuring specific peptide binding to HLA class I and class II molecules. J. Immunol. Methods 200:89-97,
22. Neisig, A, Roelse, J, Sijts, A J, Ossendorp, F, Feltkamp, M C, Kast, W M, Melief, C J, Neefjes, J J: Major differences in transporter associated with antigen presentation (TAP)-dependent translocation of MHC class I-presentable peptides and the effect of flanking sequences. J. Immunol. 154:1273-1279, 1995
23. Altman, J D, Moss, P A, Goulder, P J, Barouch, D H, McHeyzer-Williams, M G, Bell, J I, McMichael, A J, Davis, M M: Phenotypic analysis of antigen-specific T lymphocytes. Science 274:94-96, 1996
24. Groettrup, M, Ruppert, T, Kuehn, L, Seeger, M, Standera, S, Koszinowski, U, Kloetzel, P M: The interferon-gamma-inducible 11 S regulator (PA28) and the LMP2/LMP7 subunits govern the peptide production by the 20 S proteasome in vitro. J. Biol. Chem. 270:23808-23815, 1995
25. Eggers, M, Boes-Fabian, B, Ruppert, T, Kloetzel, P M, Koszinowski, U H: The cleavage preference of the proteasome governs the yield of antigenic peptides. J. Exp. Med. 182:1865-1870, 1995
26. Keymeulen, B, Ling, Z, Gorus, F K, Delvaux, G, Bouwens, L, Grupping, A, Hendrieckx, C, Pipeleers-Marichal, M, Van Schravendijk, C, Salmela, K, Pipeleers, D G: Implantation of standardized beta-cell grafts in a liver segment of IDDM patients: graft and recipients characteristics in two cases of insulin-independence under maintenance immunosuppression for prior kidney graft. Diabetologia 41:452-459, 1998
27. Roep, B O, Stobbe, I, Duinkerken, G, van Rood, J J, Lernmark, A, Keymeulen, B, Pipeleers, D, Claas, F H, De Vries, R R: Auto- and alloimmune reactivity to human islet allografts transplanted into type 1 diabetic patients. Diabetes 48:484-490, 1999
28. van der Burg, S H, Ras, E, Drijfhout, J W, Benckhuijsen, W E, Bremers, A J, Melief, C J, Kast, W M: An HLA class I peptide-binding assay based on competition for binding to class I molecules on intact human B cells. Identification of conserved HIV-1 polymerase peptides binding to HLA-A*0301. Hum. Immunol. 44:189-198, 1995
29. Liblau, R S, Wong, F S, Mars, L T, Santamaria, P: Autoreactive CD8 T cells in organ-specific autoimmunity: emerging targets for therapeutic intervention. Immunity. 17:1-6, 2002
30. Verge, C F, Stenger, D, Bonifacio, E, Colman, P G, Pilcher, C, Bingley, P J, Eisenbarth, G S: Combined use of autoantibodies (IA-2 autoantibody, GAD autoantibody, insulin autoantibody, cytoplasmic islet cell antibodies) in type 1 diabetes: Combinatorial Islet Autoantibody Workshop. Diabetes 47:1857-1866, 1998
31. Ohashi, P S, Oehen, S, Buerki, K, Pircher, H, Ohashi, C T, Odermatt, B, Malissen, B, Zinkernagel, R M, Hengartner, H: Ablation of “tolerance” and induction of diabetes by virus infection in viral antigen transgenic mice. Cell 65:305-317, 1991
32. Hunt, D F, Henderson, R A, Shabanowitz, J, Sakaguchi, K, Michel, H, Sevilir, N, Cox, A L, Appella, E, Engelhard, V H: Characterization of peptides bound to the class I MHC molecule HLA-A2.1 by mass spectrometry. Science 255:1261-1263, 1992
33. Parker, K C, Bednarek, M A, Hull, L K, Utz, U, Cunningham, B, Zweerink, H J, Biddison, W E, Coligan, J E: Sequence motifs important for peptide binding to the human MHC class I molecule, HLA-A2. J. Immunol. 149:3580-3587, 1992
34. Ruppert, J, Sidney, J, Celis, E, Kubo, R T, Grey, H M, Sette, A: Prominent role of secondary anchor residues in peptide binding to HLA-A2.1 molecules. Cell 74:929-937, 1993
35. Mo, X Y, Cascio, P, Lemerise, K, Goldberg, A L, Rock, K: Distinct proteolytic processes generate the C and N termini of MHC class I-binding peptides. J. Immunol. 163:5851-5859, 1999
36. Daniel, D, Wegmann, D R: Protection of nonobese diabetic mice from diabetes by intranasal or subcutaneous administration of insulin peptide B-(9-23). Proc. Natl. Acad. Sci. U.S.A 93:956-960, 1996
37. Martinez, N R, Augstein, P, Moustakas, A K, Papadopoulos, G K, Gregori, S, Adorini, L, Jackson, D C, Harrison, L C: Disabling an integral CTL epitope allows suppression of autoimmune diabetes by intranasal proinsulin peptide. J. Clin. Invest 111:1365-1371, 2003
38. Tian, J, Atkinson, M A, Clare-Salzler, M, Herschenfeld, A, Forsthuber, T, Lehmann, P V, Kaufman, D L: Nasal administration of glutamate decarboxylase (GAD65) peptides induces Th2 responses and prevents murine insulin-dependent diabetes. J. Exp. Med. 183:1561-1567, 1996
39. Tian, J, Clare-Salzler, M, Herschenfeld, A, Middleton, B, Newman, D, Mueller, R, Arita, S, Evans, C, Atkinson, M A, Mullen, Y, Sarvetnick, N, Tobin, A J, Lehmann, P V, Kaufman, D L: Modulating autoimmune responses to GAD inhibits disease progression and prolongs islet graft survival in diabetes-prone mice. Nat. Med. 2:1348-1353, 1996
40. Tisch, R, Wang, B, Serreze, D V: Induction of glutamic acid decarboxylase 65-specific Th2 cells and suppression of autoimmune diabetes at late stages of disease is epitope dependent. J. Immunol. 163:1178-1187, 1999
41. Elias, D, Reshef, T, Birk, O S, van der, Z R, Walker, M D, Cohen, I R: Vaccination against autoimmune mouse diabetes with a T-cell epitope of the human 65-kDa heat shock protein. Proc. Natl. Acad. Sci. U.S.A 88:3088-3091, 1991
42. Bockova, J, Elias, D, Cohen, I R: Treatment of NOD diabetes with a novel peptide of the hsp60 molecule induces Th2-type antibodies. J. Autoimmun. 10:323-329, 1997
43. van Halteren, A G, van Etten, E, de Jong, E C, Bouillon, R, Roep, B O, Mathieu, C: Redirection of human autoreactive T-cells Upon interaction with dendritic cells modulated by TX527, an analog of 1,25 dihydroxyvitamin D(3). Diabetes 51:2119-2125, 2002
44. Roelen, D L, Schuurhuis, D H, van den Boogaardt, D E, Koekkoek, K, van Miert, P P, van Schip, J J, Laban, S, Rea, D, Melief, C J, Offringa, R, Ossendorp, F, Claas, F H: Prolongation of skin graft survival by modulation of the alloimmune response with alternatively activated dendritic cells. Transplantation 76:1608-1615,
45. Woltman, A M, de Fijter, J W, Kamerling, S W, Paul, L C, Daha, M R, van Kooten, C: The effect of calcineurin inhibitors and corticosteroids on the differentiation of human dendritic cells. Eur. J. Immunol. 30:1807-1812, 2000

1. A peptide comprising amino acid residues 10-18 of the human insulin B chain (SEQ ID NO: 1) or an analogue of said peptide having 1 amino acid substitution.

2. A proteinaceous complex comprising a peptide or analogue thereof according to claim 1.

3. A proteinaceous complex according to claim 2, further comprising an HLA class I molecule or a peptide binding part, derivative and/or analogue thereof.

4. A proteinaceous complex according to claim 3 wherein the HLA class I molecule is HLA-A2.

5. A proteinaceous complex according to claim 4 wherein the HLA class I molecule is HLA-A*0201.

6. A proteinaceous complex comprising a tetramer of an HLA class I molecule and a peptide according to claim 3, optionally carrying a label.

7. A proteinaceous complex according to claim 3, further comprising a toxin.

8. A pharmaceutically acceptable composition comprising a peptide or analogue thereof as defined in claim 1.

9. A method for determining whether an individual exhibits an immune response against insulin producing cells or is at risk of developing said immune response, the method comprising determining in vitro whether a sample comprising T-cells of said individual comprises T-cells that are reactive with a peptide according to claim 1.

10. A method according to claim 9, wherein said method comprises determining whether said sample comprises T-cells that recognize an HLA class I tetramer according to claim 6.

11. A human T-cells receptor specific for a peptide or analogue thereof according to claim 1 that is present in the context of an HLA class I molecule.

12. A method of treating an individual suffering from, or at risk of suffering from, an immune response against insulin producing β-cells comprising administering an effective amount of the peptide or analogue thereof according to claim 1.

13. A method of treating diabetes mellitus comprising administering an effective amount of the composition according to claim 8 to a patient in need thereof.

14. A method for in vitro generating tolerizing dendritic cells for a peptide as defined in claim 1, comprising the step of activating said dendritic cells in the presence of a glucocorticoid receptor activating substance and loading said dendritic cells with a peptide or analogue thereof as defined in claim 1.

15. A method according to claim 14, wherein said dendritic cells are obtained by differentiating isolated peripheral blood monocytes from a subject into dendritic cells in vitro.

16. A dendritic cell produced by the method of claim 14.

17. A diagnostic kit comprising a composition, the composition comprising at least a peptide or analogue thereof as defined in claim 1.

18. A diagnostic kit according to claim 17, further comprising an HLA-class I molecule or a peptide binding part, derivative and/or analogue thereof.

19. A diagnostic kit, comprising a proteinaceous complex according to claim 2.

20. A pharmaceutically acceptable composition comprising a complex as defined in claim 2.

21. A method for determining whether an individual exhibits an immune response against insulin producing cells or is at risk of developing said immune response, the method comprising determining in vitro whether a sample comprising T-cells of said individual comprises T-cells that are reactive with a proteinaceous complex according to claim 2.

22. A method of treating an individual suffering from, or at risk of suffering from, an immune response against insulin producing β-cells comprising administering an effective amount of the proteinaceous complex according to claim 2.

23. A method of treating an individual suffering from, or at risk of suffering from, an immune response against insulin producing β-cells comprising administering an effective amount of the composition according to claim 8.
